(19) United States
(12) Patent Application Publication
(10) Pub. No.: US 2006/0074129 A1
(43) Pub. Date: Apr. 6, 2006

(54) Method for obtaining ozonized oils and vegetable fats and use of said products for pharmaceutical and cosmetic purposes

(76) Inventors: Jesus Moleiro Mirabal, Playa, Ciudad Habana (CU); Silvia Amparo Menendez Cepero, Playa, Ciudad Habana (CU); Vilfredo Felix Diaz Rubi, La Lisa, Ciudad Habana (CU); Lidia Asela Fernandez Garcia, Playa, Ciudad Habana (CU); Oscar Ernesto Ledea Lozano, Playa, Ciudad Habana (CU); Maritza Felisa Diaz Gomez, Cerro , Ciudad Habana (CU); Irene De La Mercedes Lezcano Lastre, Plaza de la Revolucion, Ciudad Habana (CU)

Correspondence Address: HALLISKY & PHILIPP, 1725 WESTLAKE AVENUE NORTH, SUITE 150, SEATTLE, WA, 98109, US

(21) Appl. No.: 10/511,025

(22) PCT Filed: Apr. 2, 2003
(86) PCT No.: PCT/CU03/00003

§ 371 Date: Apr. 28, 2005

(30) Foreign Application Priority Data
Apr. 8, 2002 (CU) 20002-0071

(51) Int. Cl. A61K 31/225; C07C 51/34; C07C 51/16

(52) U.S. Cl. 514/547; 554/133

(57) ABSTRACT

This invention is linked to the chemical, pharmaceutical and cosmetic industries, where the bactericidal, virucidal, parasiticidal and fungicidal properties of the ozonized vegetable oils and fats are considered. The emulsified vegetable oil or fat is driven to react with ozone in a bubbling reactor, covered by a jacket that makes possible to control the reaction's temperature. Ozone reacts with the double bonds present in vegetable oil or fat, giving rise to the formation of different peroxidic species, responsible for the biological action of those products. The ozonized vegetable oils and fats in those conditions are applied with satisfactory and advanced results to the treatment of diseases in diverse medical specialties, such as ophthalmology, dentistry, dermatology, gastroenterology, gynecology, parasitology, and others. Likewise, the cosmetic application of the ozonized vegetable oil and fats is described, considering their oxygenating and revitalizing properties of the skin. The results of the toxicological studies previously performed show that the products obtained according to this process do not show any cross or adverse reaction.

### TECHNICAL FIELD

This invention is according to the A61 K, CO7C and CO9K indexes of the international patents' classification and they are related to a procedure for obtaining ozonized vegetable oils and fats, as well as to the production of ointments for skin care and also to the pharmacological ATC classification, according to the anatomical, therapeutic and chemical systems. This invention consists of a new procedure for obtaining ozonized vegetable oil and fats and their medical and cosmetic applications.

### TECHNICAL BACKGROUND

The reaction of ozone with the unsaturated compounds has been widely studied (Ozonation in Organic Chemistry, Volumes 1 and 2, Philip S. Bailey, Editors: Alfred T. Blomquist and Harry H. Wasserman, Academic Press, New York, 1978) and the biological activity of the reaction's products has been acknowledged for a long time (U.S. Pat. Nos. 925,590 and 984,722). Two main groups of compounds are remarkable among the ozonized unsaturated compounds for pharmaceutical and cosmetic uses: the ozonized terpenes (U.S. Pat. Nos. 925,590, 1,081,017, 5,086,076, 5,126,376, 5,260,342, 5,270,344 and 5,364,879) and the ozonized vegetable oils (U.S. Pat. Nos. 984,722, 1,210,949, 2,356,062, 3,504,038, 4,451,480, 4,591,602, 5,183,911, RU 2040235 A, RU 2131673 C1, FR 2784388, WO 01/37829 A1 and CU 22749).

When the ozone reaction occurs with the unsaturated fatty acids that compose the triglycerides present in the vegetable oils and fats, a series of peroxidic products are formed (hydroperoxides, ozonides, diperoxides, peroxides and polyperoxides) that are responsible for the wide biological activity of those ozonized vegetable oils. The mechanism of this reaction is well defined, as well as the conditions necessary for the development of the reaction in order to enhance the preferential formation of any of those peroxidic compounds (Ozonation in Organic Chemistry, Volumes. 1 and 2, Philip S. Bailey, Editors: Alfred T. Blomquist and Harry H. Wasserman, Academic Press, New York, 1978).

The first patent on the ozonation of a vegetable oil was accepted in 1911 (U.S. Pat. No. 984,722). Its author, Alexander Twombly, carried out the ozonation of coconut oil to be used as an ointment for the treatment of nasal infections. The ozonation of that oil is carried out in liquid phase at a temperature higher than 28° C., without the use of any solvent or additive and by using medicinal oxygen for producing ozone. Ozonation is performed until reaching a stable ozone concentration in the vegetable oil of 0.5 mg of ozone by milliliters of oil.

Subsequently, there were other reports on ozonation, until consumption of all the double bonds (saturation), of different vegetable oils, such as castor oil (U.S. Pat. No. 1,210,949), olive oil (U.S. Pat. No. 2,356,062) and soybean oil (U.S. Pat. No. 3,504,038). In the first two patents, the ozonation of oil is performed in pure condition and with pharmaceutical purposes, while in the last one, Beal carries out the ozonation in aqueous medium and in the presence of aldehydes or their dimethyl acetals, to obtain high-yield aldehydes. He reaches the obtainment of the aldehydes by combining ozonation and the later reduction with hydrogen. The antecedents of Beal's work were the Otsuki's patents (U.S. Pat. No. 2,862,940) and Maggiolo's (U.S. Pat. No. 2,865,937) on the ozonation of fatty acids and their esters in aqueous medium. In the three cases, a large amount of water is used in the reaction medium (2.5-3 times in water/vegetable oil or fat). In the literature of patents, we only found the above mentioned papers where the ozonation is carried out in the presence of water and it is used as a tool for the synthesis of mono- and bi-functional aldehydes with three, six and nine carbon atoms.

From the point of view of therapeutic applications, Knox (U.S. Pat. No. 1,210,949) recommends the use of ozonized castor oil as a laxative and against intestinal infections, while Johnson (U.S. Pat. No. 2,356,062) says that ozonized olive oil and triolein have germicidal and deodorant properties, and they can be used for treating nasal or vaginal infections and for second- or third-degree burns. We must say that the consulted patents do not contain, in their text, the documented proofs of those applications and their language is mainly descriptive and general.

Other patents describe the ozonation of diverse vegetable oils, such as corn, olive and sesame oils (U.S. Pat. No. 4,451,480). This author, Richard L. DeVillez, carries out the ozonation of those vegetable oils until removing all their unsaturations, similarly to the procedures described by Knox and Johnson and recommends the use of those ozonized oils for the treatment of acne. However, he subsequently developed a method for the ozonation of jojoba oil. The reaction is performed by bubbling ozone through a glass porous disk and keeping the temperature at 50±5° C. (U.S. Pat. No. 4,591,602). Likewise, this patent fixes the oil refraction index as a method for the follow-up of the reaction and states that the ozonized oil is highly concentrated for most of the topic applications until the complete consumption of the unsaturations. That is why he uses different substances for their subsequent dilution. The ozonized jojoba oil is a product with excellent cosmetic properties because of its creamy, slightly oily consistency, its germicidal power and its soft scent. Because of the above-mentioned features, DeVillez proposes its application in cosmetics and recommends their use also for the treatment of acne and some other applications.

A patent for the ozonation procedure of vegetable oils is the one developed by Washuttl and Viebahn (DE-36067356, EP0235528, U.S. Pat. No. 5,183,911). Those authors protect the obtainment of stable ozonized vegetable oils by performing ozonation until saturation. After ozonation, they carry out an extraction process in acid medium and in the presence of a redox system (preferably of a biological redox system) for separating the short-chain aldehydes from the ozonized oil. The authors prefer, among the different vegetable oils, the olive and thistle oils, but mainly the olive oil.

Also, there are two Russian patents of ozonation procedures for vegetable oils (RU 2040235 A and RU 2131673 C1). In both patents, ozonation is carried out up to the removal of the double bonds of vegetable oils, such as olive, linseed, sunflower, almond and other oils, by bubbling an ozone-oxygen mixture through the oil. Likewise, only the peroxide index (PI) is used as a process control. The ozonized oil obtained by the procedure described in the first patent (RU 2040235 A) shows a PI of 700-900 units, but the peroxidic species formed are rapidly decomposed and the drug activity abruptly decreases. The microbiological activity of the peroxidic species, according to those aspects discussed by the authors of the second patent (RU 2131673 C1), does not exceed three months. That is why they developed a procedure where they combine the low-frequency ultrasound process (18-44 kHz) with ozonation in a bubbling reactor. With that combination, ozonized oils with higher peroxide indexes (over 1000 units) are obtained and they are stable for six months. The products they have obtained are recommended for the treatment of erysipela, ulcers, wounds, post-operatory disorders and ear infections, although, in all cases, they only present the result of a patient's evolution.

Previously, in Cuba, a patent had been registered which described the antiparasitic activity of the ozonized sunflower oil and its effectiveness, demonstrated by diverse clinical trials, in the treatment of the infections caused by Giardia lamblia (CU 22749).

Recently, Gómez et al. presented different compositions of ozonized vegetable oils mixtures, with tioctic acid and/or different substances, such as vitamins and essential aminoacids, and others (WO 01/37829 A1). Likewise, they perform the ozonation of the vegetable oils in absence of any solvent or additive and up to high levels of removal of the double bonds. They use, as a termination criterion of ozonation, the beginning of the solidification of the vegetable oil at the reaction's temperature. This termination criterion of the ozonation has the problem of its change for each type of vegetable oil being ozonized. The mixture of ozonized vegetable oils and tioctic acid are used in the treatment of gastroduodenal ulcers, in the manufacture of creams for repairing the epithelial tissue, in diet objectives or in food supplements, such as a production of enriched yoghurts or in capsules for increasing the vitality and the control of fat and body weight.

Generally, in the literature of patents, diverse processes for obtaining ozonized vegetable oils, previously discussed, are referred and they have as common characteristics the ozonation until the almost complete or complete removal of the unsaturations in oil, the ozonation of the vegetable oil with no addition of any additive or solvent and the only use of oxygen for obtaining those ozonized oils. According to the described processes, most part of the natural vegetable oils commercialized have been ozonized. Those processes show, as the most important difficulty, the low stability of the peroxidic products obtained during the process. The attempts for improving that, have caused the use of complex and expensive technologies that have only improved those products in a slight way.

Also, we can state that the germicidal properties of the ozonized vegetable oils have made possible their application in the treatment of infectious diseases. In the patents reviewed, the ozonized vegetable oils have been used in the treatment of microbial infectious of the skin (infected wounds, fistulas, acne, infected burns and ulcers) and in the treatment of nasal, ear and vaginal infections (U.S. Pat. Nos. 4,451,480, 4,591,602, 984,722, 2,356,062, 1,210,949). They have been also used in the treatment of gastroduodenal ulcers (WO 01/37829 A1) and they have been recently used in the treatment of the Giardia lamblia infection (CU 22749).

The ozonized vegetable oils and fats have been also used in cosmetics. Since the 1950s, in France, the ozonized solutions have been used as cosmetics, directly on the skin or in baths, as stimulants, purifiers, as decongestant, tranquilizers and regenerating substances of the epidermal tissue (Guillard., Dumont G. Les Nouvelles Esthetiques, November-December, 3, 1957). To those purposes, the ozonized triolein, containing around 50 volumes of ozone, was also used. The cosmetic anhydric products contained between 5 to 10% of ozonized triolein, and the triolein ozonides were the only commercial ozonides by that time (DeNavarre M. G., Wilson S. The chemistry and manufacture of cosmetics. Second Edition. Vol II-Cosmetic Materials. DD. Van Nostrand Company, INC, Princeton, N.J., p-222,1962 and FR Pat. No. 793,471).

The properties for stimulating the tissue regeneration, the oxygenation of the cells and tissues and the moderated whitening properties are added to the acknowledged germicidal activity of the products from the ozonation of unsaturated compounds, such as terpenes, fatty acids, triglycerides and vegetable oils in the cosmetic applications. (GB Pat. No. 820,463). The highly oxygenated compounds, such as the ozonized vegetable oils, favor the flexibility and the softening of the skin (DeNavarre M. G., Wilson S. The chemistry and manufacture of cosmetics. Second Edition. Vol II-Cosmetic Materials. DD. Van Nostrand Company, INC, Princeton, N.J., p-1, 1962, Ferlin H. J., Ballun A. T., Karabinos J. V. J. Am. Oil. Chemists Soc. 31, 103,1954 and Stevens F. A. J. Bacteriol. 32, 47, 1936).

Theobroma oil, as some other vegetable oils, has been widely used in cosmetic compounds, especially as a basis for ingredients with biological activity, because of its taste and odor features, in addition of having a melting point between 33 and 35° C. very close to the human body's temperature (U.S. Pat. Nos. 4,847,267; 5,849,729; 5,837,227; 4,946,832; 5,045,308). This fat is very resistant to autooxidation and keeps its high quality for a long time. Nevertheless, in the literature reviewed, there is not any reference on the use of ozonized theobroma oil for medical or cosmetic treatments.

### DESCRIPTION OF THE INVENTION

This patent discusses the constitution of a new procedure for the obtainment of ozonized vegetable oils and fats with medical, pharmaceutical and cosmetic purposes. Those ozonized oils and fats are used in different medical specialties, such as ophthalmology, dentistry, dermatology, gastroenterology, gynecology, parasitology, and others. The cosmetic creams, whose active principles are those ozonized vegetable oils and fats, are also the purpose of this invention.

The ozonation process occurs in a water—vegetable oil or fat 1-50% volume. The first new aspect described in this patent is the fact that the ozonation reaction occurs in emulsion. A stainless steel bubbling reactor is used (but it can be built in any ozone-resistant material), and it is stirred and covered by an outer jacket allowing to control the reaction's temperature by a continuous flow of water through it. The most part of the vegetable oils and fats are ozonized at temperatures between 30 and 50° C. Ozone can be obtained from medicinal oxygen and from air, but to obtain it from air it is necessary to put a washing flask with distilled water between the ozonizer and the reactor. This single step guarantees the purification of the mixture of gases from the ozonizer. The introduction of that washing flask is another new feature of this procedure. The water contained in this washing flask must be changed with the necessary frequency. The air used for generating ozone must have a dew point under −50° C. and must be fat- and powder-free. The procedure described is also valid if a piece of equipment for obtaining oxygen-enriched air (PSA) is used as source of oxygen for obtaining ozone.

The ozone generator used produces an ozone concentration between 1-15% gas volume, depending on the source of oxygen used, the gas flow, the voltage installed and the efficacy of the ozonizer. The ozone-oxygen mixture (or ozone-air) goes through the oil or fat (this last one in liquid phase) in all its volume and continuously during all the ozonation process. A gas flow (L/h)—vegetable oil or fat volume (L) between 100 and 500 is used. This high relationship makes possible to decrease the concentration of the most volatile compounds, during the ozonation process itself, when they are driven and taken out of the reactor by the gas flow. Likewise, it helps keep the emulsion during the whole process, what enhances the preferred formation of the ∝-hydroxi-hydroperoxides and delays the formation of polymers. This makes possible to obtain high indexes of peroxide and low values of viscosity. As indicators of the quality of the oils ozonized by the proposed procedure, the peroxide index (PI) can be used (measured two minutes after the contact time with potassium iodide) and the acid value (AV) and/or the concentration of aldehydes and/or the determination of viscosity, according to USP XXIV and B.P. 2000. The peroxide index is usually used as an indicator of the advancement and/or the control of the process because of its simplicity, rapidity and low cost.

The process described consists of only one unitary step, so it is simpler than the previously described procedures. Another new feature of the described procedure is that the ozonation reaction does not occur until the unsaturation present in the vegetable oil or fat is totally removed. With the proposed procedure, products with excellent germicidal properties are obtained, able to be used as drugs for the infection treatment caused by bacteria and viruses or fungi; besides, they are safe for animals and humans at the therapeutic doses.

The results from the chemical stability and the pharmacological and toxicological tests of the ozonized vegetable oils are satisfactory in our conditions. For example, the activity of the sunflower oil, ozonized according to our procedure, remains without any change for more than a year after its production, while the ozonized theobroma oil, present in different formulations, is stable for 18 to 24 months.

The diverse tests performed with ozonized sunflower oil showed the safety of this kind of products: toxicological tests (Revista CENIC, Vol 26, No. Especial, p104, 1995), -histological tests (Revista CENIC Ciencias Biológicas 20 (1-2-3), 23, 1989),—mutagenic tests (Revista CENIC Ciencias Biológicas 20 (1-2-3): 1-4, 1989),—genotoxic tests (Revista CENIC Ciencias Biológicas 29 (3): 200, 1998) and teratogenic tests (Memorias del 1^{er} Congreso Iberolatinoamericano de Aplicaciones del Ozono, Ciudad de La Habana, Ozono en Medicina 11, 1990). The tests performed with ozonized theobroma oil showed similar results:—toxicological tests (Revista CENIC, Vol. 26, No. Especial, p 105, 1995), pharmacological tests (Revista CENIC Ciencias Biológicas, Vol. 29, No 3, p 206, 1998 and Revista Mexicana de Ciencias Farmacéuticas, Vol. 33, No 3, p 30, 2002).

The germicidal properties and the stability of the vegetable oils or fats ozonized by our procedure, and particularly the ozonized theobroma and sunflower oils make possible their application for the treatment of many diseases directly caused by viruses, bacteria and fungi. They can also be used for the treatment of non-infectious diseases but where any complication by infection is present.

Considering the germicidal properties of our ozonized vegetable oils or fats, we will relate a series of new applications in the field of ophthalmology, such as the treatment of the surface keratitis, corneal ulcers, blepharitis, conjunctivitis of diverse etiology and their complications (epidemic hemorrhagic conjunctivitis, keratoconjunctivitis, and others). Those applications are shown in the examples from 3 to 7.

Other new applications, where the germicidal properties of the vegetable oils and fats ozonized by the described procedure are present, now in the field of Dentistry and diseases of the oropharyngeal cavity, are the treatment of the gingivostomatitis, the acute herpetic gingivostomatitis, the acute ulcer-necrotizing gingivitis, the infected radicular ducts, the alveolitis, the dentinal hyperesthesia, dyschromy, mucositis, periodontitis and tonsilitis. All those pathologies have as common features, the existence of infections, except dyschromy, a disease for which the ozonized sunflower oil reaches its efficacy due to the high oxidative power of the peroxidic species that compose it (mainly, the hydroxi-hydroperoxides). There is not any former reference of a clinical application of the decolorizing action of the ozonized vegetable oils. Those applications are discussed in the following examples from 8 to 17.

There are other new applications of the ozonized vegetable oils or fats according to the procedure described, also based on their germicidal properties, they are the treatment of the infections of the genitourinary tract, by the human papilloma virus (HPV) or by microorganisms of the Candida genus (e.g., Candida albicans). Those applications are detailed in examples 18 and 19.

A new application of our ozonized vegetable oils and fats is their use for the treatment of infections caused by threadworms (example 20), genital simple herpes (example 21), or by fungi (such as tynea pedis, example 22).

We also describe the utilization of ozonized sunflower oil for the treatment of gastroduodenal ulcers. Even though in the literature of patents there is an antecedent of the application of the ozonized oils for the treatment of this disease, this treatment considers the utilization of the ozonized sunflower oil—tioctic acid combination. In our case, only the ozonized sunflower oil is administered and excellent results are obtained. That is described below in example 23.

One more application of the ozonized vegetable oils and fats, but based on the anti-inflammatory activity of the peroxidic species obtained by our procedure, is the treatment of the external hemorrhoids. There is not any former reference of clinical applications of the anti-inflammatory action of the ozonized vegetable oils. This application is detailed in the example 24.

The oils ozonized by the procedure described can be also useful in the treatment of infections caused by long periods of rest and immobility, like bedsores (example 25) or in the treatment of ulcers of the lower limbs caused by chronic venous insufficiency (example 26).

The ozonized vegetable oils and fats are also applied in the field of cosmetics. Ozonized theobroma oil has been used for preparing a creamy cosmetic formulation, with revitalizing properties for the damaged skin (Example 27).

### EXAMPLES

A) Examples of Ozonation Procedure of Vegetable Oils and Fats

### Example 1

### Ozonation of Sunflower Oil

An emulsion composed by 10 liters of oil and 3 liters of distilled water is placed into the bubbling reactor. A 2 m³/h ozone-oxygen gas flow, with a concentration up to 10% ozone v/v is passed through the oil. The process goes on until the oil reaches the following indicators:—peroxide index (PI) between 600-800 units, —an acid index (AI)—lower than 15 mg/g, concentration of aldehydes—between 0.4-0.9 mmol/g and a viscosity between 100-350 mPa·s. The reactor temperature is kept at 35° C. The product is stored at a temperature between 2 and 8° C.

### Example 2

### Ozonation of theobroma Oil

The temperature of the bubbling reactor is set at 50° C. by using a thermostat and 10 kg theobroma oil and 3 liters distilled water are introduced into the reactor. A 2 m³/h ozone—air gas flow with an ozone concentration up to 5% v/v is passed through the oil. The process goes on until the oil reaches a peroxide index (PI) between 1000-1200 units and an acid index (AI) lower than 30 mg/g, The product is stored at a temperature between 2 and 8° C. in inert recipients, until the preparation of the formulations of ovules and therapeutic and/or cosmetic creams is performed.

B) Examples of the Therapeutic and Cosmetic Utilization of the Ozonized Vegetable Oils

Ophthalmology

### Example 3

### Application of Ozonized Sunflower Oil for the Treatment of Superficial Keratitis

A study was carried out in 164 patients with superficial keratitis, over 15 years old, whose diagnosis was made by biomicroscopy. Eighty patients received the ozonized sunflower oil and eighty-four received a conventional treatment. Those patients were randomly distributed and the cure criterion was the disappearance of the symptoms and the visible signs in a slit lamp, using previously sodic fluoresceine. The experimental group received a drop of ozonized sunflower oil three times a day, mydriatics and oral vitamin therapy. The conventional medication administered to the control group was based on mydriatics, iodoxuridine, vitamin therapy and oral anti-inflammatories, such as indomethacine or benzydamine. After 6 days of treatment, 70% from the experimental group were cured, only 11% were cured from the control group. After 30 days of treatment, 98% were cured from the experimental group and 62% from the control group.

### Example 4

### Application of Ozonized sunflower Oil in the Treatment of Epidemic Hemorrhagic Conjunctivitis and its Complications

A study of a group of 100 patients suffering from epidemic hemorrhagic conjunctivitis and keratitis, as one of their complications was performed. The patients received a drop of ozonized sunflower oil twice a day. In all the cases, at the beginning and at the end of the treatment, a medical ophthalmological test was performed, including an interrogatory, a measurement of the visual acuity, an eye-scope, a test in the slit lamp and dyeing with 2% sodic fluoresceine. A fast clinical improvement of the patient was obtained since the first application of the drug, with an average cure of 5 days.

### Example 5

### Application of Ozonized Sunflower Oil to Patients with Conjunctivitis of Different Etiologies

A study of 130 patients with conjunctivitis of different etiologies was performed and they received a drop of ozonized sunflower oil twice a day. A clear amelioration of the patients was observed 24 hours after the beginning of the treatment, as well as the disappearance of the symptoms on the third and fourth days. No undesirable secondary effects were observed during the treatment, what is very important considering that the usual drugs are not generally appropriate in this case, because of the possible complications that they can cause.

### Example 6

### Application of Ozonized Sunflower Oil in the Treatment of Corneal Ulcers and Keratoconjunctivitis

A study of 40 cases carrying keratoconjunctivitis and 100 cases of corneal ulcers of bacterial (20%), viral (44%) and micotic (36%) infectious etiology was performed, considered as severe and not severe, in patients between 1 and 60 years old. The diagnosis of the ulcerous corneal lesion was performed based on their biomicroscopical characteristics in a slit lamp, with corneal scraping with a lancet and seeded in bacteriological and mycotic culture, as well as in viral immunofluorescence culture media. The severity criterion of the corneal ulcer was determined by the diameter of the lesion (bigger than 3 mm) and by the presence or absence of hypopyon, and by the depth of the infiltrate. Sometimes, those pathologies are resistant to conventional treatments, they evolve by causing destroying changes in the eyeball or opacity of the cornea, with a great decrease of vision. The experimental group (50 patients) received the ozonized sunflower oil with a frequency of a drop every two hours or four times a day, for 15 days and the control group (50 patients) received a conventional treatment based on mydriatics and cycloplegics, antivirals, antibacterials and antimycotics. In the not severe ulcers, 100% were healed in both groups, but in the patients in serious condition, 61% were healed in the experimental group and only 50% in the control group, with significant differences between both groups. In keratoconjunctivitis, 95% in the experimental group was healed and 80% were cured in the control group, with significant differences between the two groups. Likewise, a remarkable improvement was observed in the cases with not extended lesions, precociously treated. On the other hand, when the ozonized sunflower oil is associated with the conventional treatment, the evolution of the disease in deep lesions of the corneal parenchyma, is better and the healing is faster.

### Example 7

### Application of Ozonized Sunflower Oil in the Treatment of Blepharitis

The sample was constituted by 40 patients suffering from blepharitis, with acute bacterial infection. Twenty patients were treated with topical ozonized sunflower oil, three times a day, by rubbing the border of the eyelid with an applicator soaked with ozonized sunflower oil, during 7 days. The 20 other patients were treated in the same way with gentamycin ointment (0.3%), 4 times a day for 7 days. Healing of 95 and 88% was obtained with ozonized sunflower oil and gentamycin, respectively, with significant differences between both groups.

Dentistry

### Example 8

### Comparative Study of the Effect of the Ozonized Sunflower Oil in Gingivostomatitis in Relation with Conventional Treatments

One hundred sixty children suffering from aphthous gingivostomatitis, between 0 and 15 years old were treated. The clinical symptoms of the children were fever, marked anorexia, salivation, gingival pain, asthenia and uneasiness of several days of evolution. The experimental group (60 children) were daily treated with touches of ozonized sunflower oil and the control groups with three different products (by following a similar procedure that the used for the experimental group): iodoxuridine (60 children), hibitane (20 children), boroglycerine (20 children). Between the third and the seventh days of treatment, the complete healing of the lesions was reached in 75% of the patients treated with ozonized sunflower oil and in 6% of the patients from the control group, with statistically significant differences (p<0,001), regarding the other control treatments applied in a similar period of time.

### Example 9

### Application of the Ozonized Sunflower Oil in the Treatment of the Acute Herpetic Gingivostomatitis

This study covered the treatment of 113 patients with antecedents of acute herpetic gingivostomatitis, and they were daily treated with ozonized sunflower oil. In 76.9% of those patients, the symptoms disappeared after a three-day treatment; in 20.4%, they disappeared on the seventh day of treatment and in 2.7%, the symptoms disappeared on the tenth day. The microorganism most frequently isolated in the lesions was the Staphylococcus aureus.

### Example 10

### Treatment of the Acute Ulcer-Necrotizing Gingivitis with Ozonized Sunflower Oil

A random phase III clinical assay was performed in a group of 48 patients suffering from acute ulcer-necrotizing gingivitis. From those patients, 24 formed the group with ozonized sunflower oil, by topical applications on the lesions, three times a day, for 7 days. The control group (24 patients) was treated with local applications of aqueous solution of sodium perborate, as rinsings, with similar periodicity to that of the group treated with ozonized sunflower oil. The tests were performed 3 or 7 days after the beginning of the treatments. In the group treated with ozonized sunflower oil, 75% of the patients were healed compared to the control group that reached 29.2%, with a significant difference (p <0,01). Regarding the signs and symptoms assessed: gingival bleeding, signs of local acute swelling and gingival pain, they disappeared more rapidly in the group treated with ozonized sunflower oil.

### Example 11

### Application of the Ozonized Sunflower Oil in the Treatment of Infected Radicular Ducts

The sample was constituted by 200 adult patients presenting radiolucid rarefaction areas, with or without fistulas in monoradicular teeth. The patients for the study were allocated at random. The sample was distributed into two groups of 100 patients each. The test group received healings with ozonized sunflower oil, by sterile cotton balls impregnated with the oil and put it in the cavity, at the entrance of the ducts. The change of the cure was performed every 48 hours. In the control group, the healing was made at the same place and similarly, using a liquid bactericide (cresophen). In this group, a similar application was performed seven days later. Radiological and clinical tests were carried out to the patients at the start and at the end of the treatment. In the group treated with ozonized sunflower oil, the results were better, with 91% of improvement compared to the control group (55%) with significant differences (p<0,01). 88 and 5% of the patients healed with ozonized sunflower oil and cresophen, respectively, showing significant differences between both groups. Patients treated with ozonized sunflower oil needed two or three visits to the doctor's office, while most of the healed patients of the control group required four to six visits to the doctor's office.

### Example 12

### Application of the Ozonized Sunflower Oil in the Treatment of Alveolitis

The ozonized sunflower oil was used as the only drug in the treatment of alveolitis. The results were compared to those corresponding to a control group, where alvogil was used as local treatment, besides applying an oral antibiotic. The sample was formed by 100 adult patients, randomly distributed into two groups, with 50 patients each. Healings were performed every 72 hours and visits to the doctor were carried out as required. The healing criterion considered was the formation of a healing tissue and the decrease or elimination of the pain. The healing was reached in 43% of the patients treated with ozonized sunflower oil and in 41% the patients treated with alvogil, without any significant differences between both groups. However, patients treated with ozonized sunflower oil healed most rapidly and they only required two or three visits to the doctor, regarding the patients healed with alvogil that required four to six visits to the doctor.

### Example 13

### Application of the Ozonized Sunflower Oil in the Dentinal Hyperesthesia

In this study, the sample was formed by 100 patients, randomly distributed into two groups: 50 patients treated with ozonized sunflower oil and 50 were treated with placebo (non-ozonized oil). The drug was topically applied every day. In the first consultation, after the beginning of the treatment, 56% of the patients treated with ozonized sunflower oil, improved their symptoms, while that did not occur in the patients treated with placebo. In the second consultation, 10% healing was reached and 70% of the patients improved from the cases treated with ozonized sunflower oil, whereas in the placebo group, only 20% of improvement was obtained. In the third and last consultations, 91% healing was reached and 9% improvement in the patients treated with ozonized sunflower oil; in the placebo group, only 10% healing was reached, 30% improvement and 60% remained in the same condition. These results indicate that the sunflower oil (the vehicle used in this case) confers certain protection to the dentine, and this may cause the little healing or improvement observed in the placebo group. Nevertheless, the results achieved in the group treated with ozonized sunflower oil were higher than those of the placebo group with statistically significant differences.

### Example 14

### Dyschromy Treated with Ozonized Sunflower Oil

A sample of 30 patients with dyschromy were studied, 15 of those patients were treated with ozonized sunflower oil and 15 with sodium perborate and acetone. The vital teeth with dyschromy were excluded and only the non-vital teeth subject to pulptomy were treated. The whitening was reached after a five-day treatment, with similar results for both treatments.

### Example 15

### Effectiveness of the Ozonized Sunflower Oil in Cytostatic-Induced Mucositis in Children

A study was performed with 36 cancer-carrier children suffering from mucositis. The children were distributed into two groups: one group treated with ozonized sunflower oil and the other with the conventional therapies, in this case, an hibitane+nistatine combination. In 63% of the patients treated with the traditional therapies, the ulcers disappeared after 8 days of treatment, using six daily healings, whereas in the group treated with ozonized sunflower oil, using three daily healings, after a maximum of 5 days, 84% achieved a complete healing. The results showed that the ozonized sunflower oil has a clinical effectiveness higher than hibitane and nistatine in the treatment of mucositis.

### Example 16

### Application of the Ozonized Sunflower Oil in Periodontitis

In this study, we used ozonized sunflower oil in order to evaluate their effect on the treatment of moderate simple periodontitis and for preventing its recidivation. A random, controlled and single-blind phase III clinical trial was performed in 84 patients, older than 35 years, from both sexes. Ozonized sunflower oil was topically applied to 42 patients on the operated area and on the 7^{th}, 14^{th} and 21^{st} days after operation on the adjacent periodontal tissues. The control group was formed by 42 patients that received the conventional treatment with chlorhexidine (aqueous solution 0.2%). An analytic index of hygiene, clinical and radiographical tests and microbiological controls was applied to the patients, at the beginning, on the 21^{st} , 90^{th} and 180^{th} days and with intervals of 1 month until 9 months after operation. The effectiveness of the treatment after 180 days was considered as: good (satisfactory clinical and microbiological assessments) in 98% of the patients of the group treated with ozonized sunflower oil and in 78% of the control group; fairly good (some of the assessments were not satisfactory) for 2 and 17%, respectively, and in the category of bad (both assessments were not satisfactory) only 5% appears in the control group. Recidivation was more frequent in the control group (15%) than in the group treated with ozonized sunflower oil (5%). In general, the best clinical results (best evolution and healing during the study) and microbiological results were obtained in the group treated with ozonized sunflower oil, and also a lower percent of recidivation was found in that group. No side effects were observed.

### Example 17

### Application of Ozonized Sunflower Oil in Acute Tonsilitis

Fifteen patients suffering from acute tonsilitis were studied and daily treated with ozonized sunflower oil in the oropharyngeal area for a week. Microbiological controls (pharyngeal exudate) and physical tests of the oropharyngeal area were performed to those patients at the beginning and at the end of the treatment. Among the microorganisms in the first exudate we found Streptococcus pyogenes, Haemophylus influenzae, Bordetella pertussis, and others. At the end of the treatment, all patients were cured, taking into account the microbiological and clinical tests performed.

Other medical and cosmetic applications of the ozonized vegetable oils and fats.

### Example 18

### Ozonized Sunflower Oil in the Treatment of the Infection Caused by the Human Papilloma Virus

Sixteen women with the human papilloma virus (HPV) in the vagina or in the cervix were studied and treated with embrocations of ozonized sunflower oil on the affected areas, using the speculum for the curing. The treatment was daily performed for 15 days. The results, by colposcopy and cytology, showed an effectiveness of 94%.

### Example 19

### Application of Ozonized theobroma Oil Ovules in the Treatment of Vulvovaginal Candidiasis

Ninety women, 17 to 51 years old, with diagnosis of vulvovaginal candidiasis were studied. Patients were allocated at random in three groups: 30 were treated with ovules containing 10% ozonized theobroma oil, 30 were treated with Nistatine and 30 with Clotrimazole. To all patients, a vaginal exudate was performed at the beginning and at the end of the treatment, six days after the application of the last ovule. Also, an interrogation and a physical test were performed to them. The clinical symptoms of those patients were pruritus, burning and leucorrhea with higher prevalence. By comparing the groups, the symptoms and signs disappeared in a higher percent between the first and the fourth days of treatment for the groups using ozonized oil ovules and Clotrimazole and between the ninth and twelfth days for the Nistatine group. Taking into account the final exudate, a healing of 97% was obtained for the patients treated with ozonized theobroma oil ovules, 80% for the group treated with Clotrimazole and 50% for the patients treated with Nistatine. The effectiveness of the ozonized theobroma oil ovules, in the vulvovaginitis by Candida albicans, was proved. No side effects were found.

### Example 20

### Application of Ozonized Sunflower Oil in the Infection Caused by Oxyuris,

The germicidal effect of the ozonized sunflower oil was proved in the infection by nematodes, specifically in the elimination of Enterobius vermicularis or infection caused by oxyuris. Fifteen children, suffering from this nematode were treated with embrocations of ozonized sunflower oil, once a day, in the perianal region during 3 days. Two weeks later the treatment was repeated. At the end of the treatment all the children were free from oxyuris.

### Example 21

### Application of the Ozonized theobroma Oil in the Treatment of Recidivating Genital Herpes Simplex

Forty patients suffering from genital recidivating herpes simplex were randomly distributed into two groups of study, with 20 patients each. The experimental group was treated topically with an ointment containing 20% ozonized theobroma oil, twice a day, for one year and the control group received applications of physiological saline solution and zinc lotion, with a similar plan of treatment. In the experimental group, 10 patients did not suffer recidivations during the year of study and in the other 10 patients, the recidivations were not so frequent and the onset duration was decreased. In the control group, all the patients manifested that they had recidivations, only in two cases they were less frequent, but their duration did not decrease.

### Example 22

### Application of Ozonized theobroma Oil in the Treatment of Tynea Pedis

Fifty patients with a diagnosis of tynea pedis, randomly distributed into two groups of study, 25 patients in each group, were studied. The experimental group was treated with an ointment containing 20% ozonized theobroma oil, for 6 weeks, twice a day and the control group was treated with Whitfield ointment with no sulfur with a similar plan of treatment. The healing criterion was the presence of negative microbiological exudate. A healing of 85 and 20% in the experimental and control groups, respectively, with significant differences between both groups was obtained.

### Example 23

### In Vitro Study of the Effectiveness of the Ozonized Sunflower Oil on Helicobacter pylori

In the last years, a growing number of patients were found, carrying Helicobacter pylor, as some of the causing agents of the presence of gastroduodenal ulcer. An in vitro study of samples of the mucose of the digestive system contaminated by Helicobacter pylori and treated with ozonized sunflower oil was performed. The effectiveness of the ozonized sunflower oil was assessed by the urease test. No growth of Helicobacter pylori was observed in any of the samples treated with ozonized sunflower oil.

### Example 24

### Application of Ozonized Sunflower Oil in the Treatment of External Hemorrhoids

Considering the anti-inflammatory nature of the ozonized sunflower oil, as well as its oily consistency, a study was performed in 200 patients suffering from external hemorrhoids. The sample was randomly distributed into two groups of 100 patients each. The experimental group was treated with embrocations of ozonized sunflower oil, twice a day. The control group was also treated with Protolog ointment, twice a day. A disappearance of the symptoms and also a regression of the lesions were reached much faster in the group treated with ozonized sunflower oil than in the control group.

### Example 25

### Application of the Ozonized Sunflower Oil in the Treatment of Bedsores

Twenty patients suffering from bedsores in the sacral region were studied and randomly distributed into two groups of 10 patients each. The experimental group was treated with ozonized sunflower oil, twice a day, and the control group was treated with ointments, according to the germ present, with a similar plan of treatment. All the patients succeeded in the healing of their wounds. In the group treated with ozonized sunflower oil, the time of healing was shorter and it was not necessary to perform any antibiogram, because of its wide germicidal power.

### Example 26

### Application of Ozonized Sunflower Oil in the Treatment of Lower Limb Ulcers Caused by Chronic Venous Insufficiency

A study was performed with 20 patients with lower limb ulcers caused by chronic venous insufficiency with less than five years of evolution. Both groups were treated with venous rest, hyposodic diet and analgesic drugs. Besides, a mechanical disinfection with benzalconium chloride 1/5000 was performed twice a day. After disinfection, ozonized sunflower oil was applied to the experimental group and antibiotic ointments, according to the isolated germ, were applied to the control group. An amelioration of the inflammatory signs after 72 hours and the appearance of granulation tissue after the fifth day were observed in the experimental group, whereas in the control group, both the evolution and the disappearance of signs and symptoms lasted more.

### Example 27

### Application of a Cosmetic Cream Prepared from Ozonized theobroma Oil for Cutaneous Involution

The effectiveness of the product was assessed in 10 women, 25 to 50 years old. The cream was applied on the previously washed face, twice a day for six months. The beneficial effect, as for slight wrinkles, coloration and hydration of the skin was assessed in 70% of the treated women.

Advantages of the proposed solution:
The described process consists of an only unitary step, so it is simpler than the previously described processes.
The ozonation reaction is performed partially, it is not necessary to remove all the unsaturations present in the vegetable oil or fat.
By this proposed procedure, products with excellent germicidal properties are obtained and they make possible their utilization for the treatment of infections caused by bacteria, viruses, parasites or fungi.
The obtained products are characterized by being innocuous for animals and men at the therapeutical doses.
Utilization of the obtained products for the elaboration of cosmetic creams with oxygenating and revitalizing properties for the damaged skin.
The obtained products are characterized by being stable for more than 18 months at 2-8° C.

1-21. (canceled)

22. A process for the obtainment of ozonized oils and fats in a bubbling reactor, characterized by the ozonation in liquid phase of an emulsion of water in vegetable oil or fat, in a 1-50% relation in volume, by passing the ozone-oxygen or ozone-air gas mixtures through it, at a controlled temperature between 30-50° C., employing an gas flow/vegetable oil or fat volume relation between 100 and 500, and using as quality parameters: peroxide (PI) and acid (AI) indexes, and/or concentration of aldehydes and/or viscosity.

23. A procedure according to claim 22, characterized by the use of a washing flask with distilled water between the ozonator and the reactor, when air or oxygen-enriched air are used as a source of oxygen for obtaining ozone.

24. A procedure according to claim 22, characterized by the mainly formation of vegetable oil or fat α-hydroxi-hydroperoxides with a PI between 200-1200 units.

25. A medical product obtained from ozonation of sunflower oil, according to claim 22, characterized by presenting a PI between 200-800 units, an AI lower than 15 units, concentration of aldehydes between 0.4-0.9 mmol/g and viscosity between 100-500 mPa·s.

26. A product with biological activity, obtained from the ozonation of theobroma oil, according to claim 22, characterized by a PI between 1000-1200 units and an AI lower than 30 units.

27. Use of any ozonized oil or fat, according to claim 22, for the treatment of diverse ophthalmologic diseases, such as: superficial keratitis, conjunctivitis of different etiologies, epidemic hemorrhagic conjunctivitis and others, keratoconjunctivitis, corneal ulcers and blepharitis, characterized by the use of an ozonized vegetable oil or fat with a PI between 200-400 units and an AI lower than 10 units.

28. Use of any ozonized vegetable oil or fat, according to claim 22, for the treatment of the following stomatological or oropharyngeal cavity diseases, such as: acute herpetic gingivostomatitis, infected radicular ducts, alveolitis, dentinal hyperesthesia, dyschromia, cytostatic-induced mucositis, periodontitis or tonsilitis, characterized by the utilization of an ozonized vegetable oil or fat with a PI between 600-800 units and an AI lower than 15 units.

29. Use of any ozonized vegetable oil or fat, according to claim 22, for the treatment of genital urinary tract diseases, such as the infection by human papilloma virus, herpes simplex and vulvovaginal candidiasis, characterized by the utilization of an ozonized vegetable oil and fat with a PI between 600-800 units and an AI lower than 15 units, a cream containing between 20-50% weight of ozonized vegetable oil or fat and ovules containing between 5-40% weight of ozonized vegetable oil or fat, respectively.

30. Use of any ozonized vegetable oil or fat, according to claim 22, for the treatment of oxyuris, characterized by the utilization of an ozonized vegetable oil or fat with PI between 600-800 units and an AI lower than 15 units.

31. Use of any ozonized vegetable oil or fat, according to claim 22, for the treatment of helicobacter pylori infections, characterized by the administration of capsules or drops containing an ozonized vegetable oil with a PI between 600-800 units and an AI lower than 15 units.

32. Anti-inflammatory action of the ozonized vegetable oils and fats, according to claim 22, through their application in the treatment of external hemorrhoids, characterized by the use of an ozonized vegetable oil or fat with a PI between 600-800 units and an AI lower than 15 units.

33. Use of any ozonized vegetable oil or fat, according to claim 22, for the treatment of bedsores and lower limb ulcers caused by chronic venous insufficiency, characterized by the utilization of an ozonized vegetable oil with a PI between 600-800 units and an AI lower than 15 units.

34. Use of any ozonized vegetable oil or fat, according to claim 22, for cosmetic use as a revitalizing agent of the damaged skin, characterized by the utilization of a cream containing between 5-30% weight of ozonized vegetable oil or fat.
